(19) United States
(12) Patent Application Publication
(10) Pub. No.: US 2023/0201562 A1
(43) Pub. Date: Jun. 29, 2023

(54) GUIDEWIRE GUIDE SEAL FOR IMPLANTABLE MEDICAL DEVICES

(71) Applicant: TC1 LLC, St. Paul, MN (US)

(72) Inventors: Michael L. Green, Pleasanton, CA (US); Stephen D. Pacetti, St. Paul, MN (US)

(21) Appl. No.: 18/146,105

(22) Filed: Dec. 23, 2022

Related U.S. Application Data
(60) Provisional application No. 63/294,625, filed on Dec. 29, 2021.

(51) Int. Cl. A61M 60/148; A61M 60/13

(52) U.S. Cl. CPC A61M 60/148; A61M 60/13

(57) ABSTRACT

A septum for use in a catheter pump is provided herein. The septum includes a first elastomeric septa disc. The first elastomeric septa disc is punctured. The puncture forms an aperture in the first elastomeric septa disc. The aperture is passable by a guidewire.

### CROSS-REFERENCE TO RELATED APPLICATIONS

This application claims the benefit of priority to U.S. Provisional Patent Application No. 63/294,625, filed Dec. 29, 2021, the entire contents of which are hereby incorporated by reference herein.

### BACKGROUND

### a. Field of the Disclosure

The present disclosure relates generally to implantable medical devices, and more specifically, relates to guidewire guides for use in implantable medical devices.

### b. Background

Heart disease is a major health problem that has a high mortality rate. Physicians increasingly use mechanical circulatory support systems for treating heart failure. The treatment of acute heart failure requires a device that can provide support to the patient quickly. Physicians desire treatment options that can be deployed quickly and minimally-invasively.

A Percutaneous Heart Pump (PHP) system is one example of a ventricular assist device that may be used during high-risk percutaneous coronary interventions (PCI) performed electively or urgently in hemodynamically stable patients with severe coronary artery disease, when a heart team, including a cardiac surgeon, has determined high-risk PCI is the appropriate therapeutic option. Use of the PHP system in these patients may prevent hemodynamic instability, which can result from repeat episodes of reversible myocardial ischemia that occur during planned temporary coronary occlusions and may reduce pre-and post-procedural adverse events. PHP systems may also be used to treat cardiogenic shock in certain circumstances.

In at least some embodiments, the PHP system includes a distal septum to prevent blood from entering a fluid lumen of a catheter of the PHP system. In such embodiments, a guidewire guide (GWG) may extend through a distal septum for a period of time (e.g., while the PHP system is being stored), which may impact the sealing capabilities of the distal septum. Accordingly, it would be desirable to provide a GWG that facilitates reducing impacts on the sealing capabilities of the distal septum.

### BRIEF SUMMARY OF THE DISCLOSURE

In one aspect of the present disclosure a septum is disclosed for use in a catheter pump. The septum includes a first elastomeric septa disc. The first septa disc is punctured to form an aperture in the first elastomeric disc. The aperture is passable by a guidewire.

In another aspect of the present disclosure, a catheter assembly is disclosed for use in a percutaneous heart pump. The catheter assembly includes a pump assembly that includes an impeller, an impeller tip positioned distal of the impeller, and a distal septum positioned between the impeller and the impeller tip. The catheter assembly also includes a flexible atraumatic tip (FAT) positioned distal of the pump assembly. The catheter assembly also includes a guidewire guide (GWG) coupled between the pump assembly and the FAT where a proximal end of the GWG is positioned distal of the distal septum.

In yet another aspect of the present disclosure, a method of assembling a catheter assembly for use in a percutaneous heart pump (PHP) is disclosed. The method includes coupling a pump assembly to the catheter assembly where assembling the pump assembly includes positioning an impeller in the pump assembly. Assembling the pump assembly also includes positioning an impeller tip in the pump assembly at a position distal of the impeller. Assembling the pump assembly also includes positioning a distal septum between the impeller and the impeller tip. Assembling the catheter assembly also includes positioning a flexible atraumatic tip (FAT) distal of the pump assembly. Assembling the catheter assembly also includes coupling a guidewire guide (GWG) between the pump assembly and the FAT, where the proximal end of the GWG is positioned distal of the distal septum.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 illustrates one embodiment of a pump assembly positioned in the heart for operation.

FIG. 2 is a perspective cross-sectional view of a catheter assembly including an alternative embodiment of a GWG, according to one embodiment.

FIG. 3A shows a plan view of septa discs with GWG aperture alignment.

FIG. 3B shows a plan view of septa discs with disc alignment.

FIG. 4A is a perspective view of septa discs with spatial separation.

FIG. 4B is a perspective view of septa discs conjoined.

FIG. 5A is a plan view of three septa discs with GWG aperture alignment.

FIG. 5B is a plan view of three septa discs with disc alignment.

FIG. 6A illustrates one embodiment of a GWG aperture in a line pattern.

FIG. 6B illustrates an aperture in a Y pattern.

FIG. 6C illustrates an aperture in a cross pattern.

Corresponding reference characters indicate corresponding parts throughout the several views of the drawings.

### DETAILED DESCRIPTION OF THE DISCLOSURE

The disclosure provides systems and methods for guidewire guides (GWGs) for use in a medical system. In various embodiments disclosed herein, a GWG may be configured to receive a guidewire therethrough. A clinician may maneuver the guidewire to the heart through the patient's vasculature. The clinician may then advance a distal portion of a catheter assembly over the guidewire, using the GWG, to position the distal portion (e.g., including an impeller) in a chamber of the heart. In some embodiments, the GWG may include a central lumen formed along at least a portion of the length of the catheter assembly. The clinician may then withdraw the guidewire from the catheter assembly.

Additional embodiments of this disclosure are directed to apparatuses for improving resistance of fluid flow in the apparatus. For example, in the operative device, a septum may be included at a distal portion of the apparatus through which the GWG is threaded. In particular, various embodiments disclosed herein generally relate to various configurations for a septum at a distal end of a GWG in a catheter assembly. Accordingly, the septum may be comprised of one or more separate septa discs. Each septa disc may be punctured during catheter assembly. Accordingly, the septa discs may include openings or apertures of varying shape and size through which a guidewire or guidewire guide may pass through.

In the exemplary embodiment, the septa discs include offset openings respective of one another. When housed within the catheter assembly, the septa discs are in alignment and, accordingly, the openings in each respective disc are disaligned from each other respective septa disc.

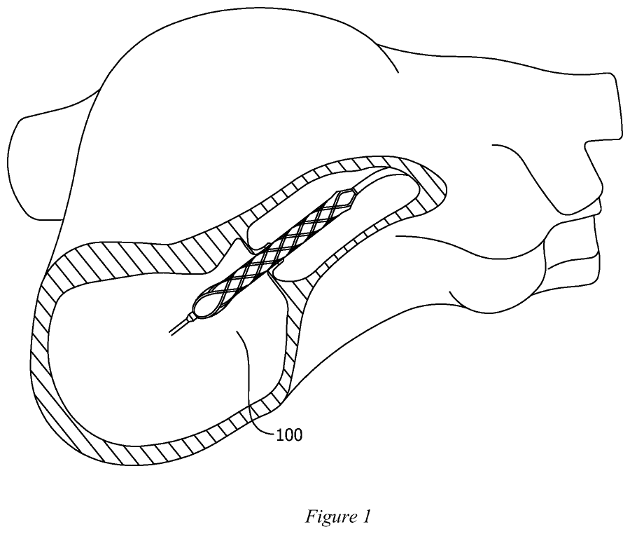

FIG. 1 illustrates one embodiment of a pump assembly 100 positioned in the heart for operation. Pump assembly 100 is fixed at a distal portion of a catheter assembly, and is placed in the left ventricle (LV) of the heart to pump blood from the LV into the aorta. Pump assembly 100 may be used in this way to treat patients with a wide range of conditions, including cardiogenic shock, myocardial infarction, and other cardiac conditions, and also to support a patient during a procedure, such as percutaneous coronary intervention. One convenient manner of placement of the distal portion of pump assembly 100 in the heart is by percutaneous access and delivery using the Seldinger technique or other methods familiar to cardiologists. Various guide features disclosed herein enable pump assembly 100 to be advanced over a guidewire to the heart. These approaches enable pump assembly 100 to be used in emergency medicine, a catheter lab, and in other non-surgical settings. Modifications may also enable pump assembly 100 to support the right side of the heart. Example modifications that may be used for right side support include providing delivery features and/or shaping a distal portion that is to be placed through at least one heart valve from the venous side, such as discussed in U.S. Pat. Nos. 6,544,216 and 7,070,555 and in U.S. Patent Publication No 2012/0203056, all of which are incorporated herein by reference for all purposes in their entirety.

Embodiments of pump assemblies, such as pump assembly 100 (shown in FIG. 1) of this disclosure may be configured with a motor that is capable of coupling to (and in some arrangements optionally decoupling from) a catheter assembly. For example, access may be provided to a proximal end of the catheter assembly prior to or during use. In one embodiment, pump assembly 100 is delivered over a guidewire (not shown). In some embodiments, the guidewire may be conveniently extended through the entire length of the catheter assembly and out of a proximal portion thereof that is completely enclosed in a coupled configuration. For this approach, connection of the proximal portion of the catheter assembly to a motor may be completed after the guidewire has been used to guide the operative device of pump assembly 100 to a desired location within the patient (e.g., to a chamber of the patient's heart). In other embodiments, the guidewire may be inserted through other types of guide features to guide pump assembly 100 to the heart. For example, in other embodiments, there may be no central lumen extending from proximal end to the distal end of pump assembly 100. Rather, the guidewire may be inserted along the side of the catheter assembly or along a short central lumen or a removable lumen.

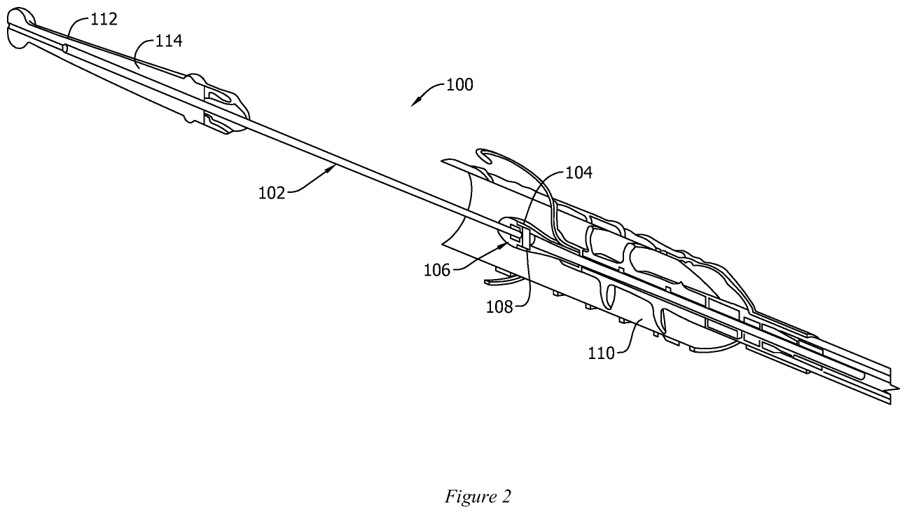

FIG. 2 shows a perspective cross-sectional view of a catheter assembly including an embodiment of a pump assembly 100 (shown in FIG. 1), according to one embodiment. Pump assembly 100 includes a guidewire guide (GWG) 102, a proximal end 104 of GWG 102, an impeller tip 106, a distal septum 108, a cannula 110, a flexible atraumatic tip (FAT) 112, and a distal end 114 of GWG 102. GWG 102 is flexible and resilient to facilitate (a) not kinking as pump assembly 100 is delivered to the left ventricle of a patient's heart and (b) not increasing a rigid length of pump assembly 100.

GWG 102 may be made of flexible alloys, such as nitinol, a grade of stainless steel, and/or advanced polymers such as polyamide, HDPE, LLDPE, FEP, PET, Pebax, etc. Any of these materials may include a pattern cut into them to improve flexibility and prevent kinking. GWG 102 may also include a reinforced sheath (e.g., a thin braid structure) that also improves flexibility and prevents kinking (particularly in embodiments where GWG 102 rotates during operation of pump assembly 100).

As shown in FIG. 2, proximal end 104 of GWG 102 is located distal of distal septum 108. Accordingly, GWG 102 does not extend across distal septum 108. Accordingly, in some embodiments, by permanently including GWG 102 in pump assembly 100, the only time distal septum 108 is pierced is when a user (e.g., a clinician) passes a guidewire through GWG 102 prior to a procedure. The guidewire may subsequently be removed, for example, as soon as pump assembly 100 is delivered inside the patient, thereby allowing distal septum 108 to be sealed quickly (e.g., in less than one hour). As explained in more detail below, distal septum 108 may include two or more septa to facilitate sealing.

As cannula 110 is sheathed, FAT 112 moves distally, for example, by approximately 0.25 inches, relative to impeller tip 106. To accommodate this, in the embodiment shown in FIG. 2, distal end 114 of GWG 102 is coupled to FAT 112 using a slip fit. Further, in the embodiment shown, GWG 102 is coupled to FAT 112, such that GWG 102 is capable of rotating or spinning relative to FAT 112 (and rotating with the impeller) during operation of pump assembly 100. In other embodiments, where GWG 102 is not rotatable or spinnable with the impeller, at least one bearing (not shown) is included in pump assembly 100, such that the at least one bearing is configured to secure GWG 102 inside pump assembly 100 and allow the impeller to rotate relative to GWG 102.

In some embodiments, before inserting pump assembly 100 into a patient, a clinician may insert a guidewire through the patient's vascular system to the heart to prepare a path for the operative device to the heart. In some embodiments, pump assembly 100 may include a GWG tube that may pass through a central internal lumen of pump assembly 100 from proximal guidewire opening in the distal end of FAT 112. The GWG tube may be pre-installed in pump assembly 100 to provide the clinician with a preformed pathway along which to insert a guidewire. Thus, in this embodiment, the guidewire may be advanced through a central lumen extending through the length of pump assembly 100. Other embodiments may include different types of guide features, as explained herein.

In one approach, the guidewire is first placed in a conventional way, e.g., through a needle into a peripheral blood vessel, and along the path between that blood vessel and the heart and into a heart chamber (e.g., into the left ventricle). Thereafter, a distal end opening of pump assembly 100 and/or a GWG tube may be advanced over a proximal end of the guidewire to enable delivery to pump assembly 100. After the proximal end of the guidewire is urged proximally within pump assembly 100 and emerges from a proximal guidewire opening and/or the GWG tube, pump assembly 100 may be advanced into the patient. In one method, the guidewire is withdrawn proximally while holding the catheter assembly.

Alternatively, the clinician may insert the guidewire through a proximal guidewire opening and urge the guidewire along the GWG tube until the guidewire extends from a distal guidewire opening (not shown) in the distal end of pump assembly 100. The clinician may continue urging the guidewire through the patient's vascular system until the distal end of the guidewire is positioned in the desired chamber of the patient's heart. A proximal end portion of the guidewire may extend from proximal the guidewire opening. Once the distal end of the guidewire is positioned in the heart, the clinician may maneuver a pump assembly over the guidewire until pump assembly reaches the distal end of the guidewire in the heart. The clinician may remove the guidewire and the GWG tube. In some embodiments, the GWG tube may also be removed before or after the guidewire is removed. Other embodiments for inserting a guidewire through different types of guide features are explained in more detail below.

After removing at least the guidewire, the clinician may activate a motor to rotate the impeller and begin operation of pump assembly 100. In some instances, when using the guidewire to guide the operative device to the heart, a central lumen or tube (e.g., a GWG) is typically formed to provide a path for the guidewire.

As shown in FIG. 2, a cannula housing may be coupled to a distal tip member at distal end 114. The distal tip member may be configured to assist in guiding the operative device of the catheter assembly (e.g., a pump assembly) along the guidewire. As described above, one exemplary distal tip member may be formed of a flexible material and may have a rounded end to prevent injury to the surrounding tissue. If the distal tip member contacts a portion of the patient's anatomy (such as a heart wall or an arterial wall), the distal tip member will safely deform or bend without harming the patient. The distal tip member may also serve to space the operative device away from the tissue wall.

In addition, GWG 102 may extend through a central lumen of pump assembly 100. Thus, GWG 102 may pass through the impeller shaft (not shown in FIG. 2) and a lumen formed within the distal tip member at distal end 114. GWG 102 may include the central lumen extending throughout the length of pump assembly 100. GWG 102 may extend distally past distal end 114. As explained above, in various embodiments, the clinician may introduce a proximal end of GWG 102 into distal end 114, which extends distally beyond the distal tip member. In some embodiments, once GWG 102 has been inserted into the patient, GWG 102 may be removed from the catheter assembly.

In some embodiments, GWG 102 does not extend distally past the end of the distal tip member at distal end 114. Rather, the central lumen passing through the distal tip member may include a proximal lumen and a distal lumen. The proximal lumen may have an inner diameter larger than an inner diameter of the distal lumen. A stepped portion or shoulder may define the transition between the proximal lumen and the distal lumen. The inner diameter of the proximal lumen is sized to accommodate GWG 102 as it passes through a portion of the distal tip member. However, the inner diameter of the distal lumen is sized to be smaller than the outer diameter of GWG 102 such that GWG 102 is too large to pass through the distal lumen of the distal tip member. In some embodiments, the thickness of GWG 102 may be made smaller than the height of the stepped portion or shoulder (e.g., smaller than the difference between the inner diameter of the proximal lumen and the inner diameter of the distal lumen). By housing GWG 102 against a shoulder, the shoulder may protect the outer coating of a guidewire when the guidewire is inserted proximally from the distal lumen to the proximal lumen.

Some embodiments may assist the clinician in inserting the guidewire into the distal end of pump assembly 100. For example, GWG 102 may be inserted through the central lumen of pump assembly 100. For example, GWG 102 may pass distally through a portion of the motor, the catheter body, the pump assembly, and cannula 110, and through the proximal lumen of the distal tip member. GWG 102 may be urged further distally until the distal end of GWG 102 reaches the shoulder. When the distal end of GWG 102 reaches the shoulder, the shoulder may prevent further insertion of GWG 102 in the distal direction (e.g., the shoulder may have a smaller diameter that the diameter of GWG 102). Because the inner diameter of the distal lumen is smaller than the outer diameter of GWG 102, the distal end of GWG 102 may be disposed just proximal of the. The shoulder may be made of a flexible material, which may result in expansion when a distal end of GWG 102 is pushed against the shoulder. In some embodiments, the shoulder may be coupled to a rigid ring that forms a non-deformable ledge. The ring facilitates maintaining the diameter of the shoulder and prevents GWG 102 from expanding the shoulder and moving beyond the shoulder.

The clinician may insert the proximal end of the guidewire proximally through a distal lumen passing through a rounded tip at the distal end of distal tip member. Because the distal tip member is flexible, the clinician may easily bend or otherwise manipulate the distal end of the distal tip member to accommodate the small guidewire. Unlike GWG 102, which may be generally stiffer than the distal tip member, the clinician may easily deform the distal tip member to urge the guidewire into the distal lumen. Once the guidewire is inserted into the distal lumen, the clinician may urge the guidewire proximally past stepped portion or shoulder and into larger GWG 102, which may be positioned within proximal to the lumen. Furthermore, since most commercial guidewires include a coating (e.g. a hydrophilic or antimicrobial coating, or PTFE coating), GWG 102 and the shoulder advantageously avoids damaging or removing the coating. When the wall thickness of GWG 102 is less than the height of the step or shoulder, the shoulder may substantially prevent GWG 102 from scraping the exterior coating off of the guidewire. Instead, the guidewire easily passes from the distal lumen to the proximal lumen. The guidewire may then be urged proximally through the impeller and catheter assembly until the guidewire protrudes from the proximal end of the system, such as through a proximal guidewire opening.

The GWG features may include a central lumen passing through pump assembly 100 along its length. In some embodiments, it may be desirable to omit the central lumen through pump assembly 100. For example, removing the central lumen from the drive cable and motor assembly may advantageously simplify the manufacturing process and may reduce the profile (e.g., diameter) of pump assembly 100.

As described herein, GWG 102 may include various embodiments of a distal portion of a GWG 102. As described above, GWG 102 may include a GWG tube (also referred to herein as a hypotube). The hypotube may include a relatively rigid hypodermic tubing made of stainless steel. The distal tip of the hypotube may be formed in different shapes and/or with varying angles. For example, the distal end of the hypotube may be narrower than other portions of the hypotube. The hypotube may include a polymeric sleeve or flexible tubular lumen, thereby making the distal end of the GWG tube (which contacts the inner lumen of sealing septum 108 and/or rigid transition, as shown in FIG. 2) more conformable and expandable. The varying shapes, material compositions, construction, coatings, and surface of the hypotube may be designed to reduce abrasion when in contact with components of pump assembly 100, such as distal septum 108.

The hypotube may include a sleeve (e.g., a polymeric sleeve or flexible tubular lumen) that may be extruded or constructed from any of a number of polymeric materials similar to a tip of a balloon, such as polyamide elastomers (similar to inner members of balloon catheters), polyethylene copolymers, PTFE blends, or combination thereof. The sleeve may be compliant, expandable, electrospun, braided, mesh-like, webbing-like, and/or similar to a wrap or tubular lumen. The sleeve may also be coated to further reduce friction between contact components such as distal septum 108. The coating may include silicone or mineral oil based materials, hydrophobic materials, hydrophilic materials, or other materials suitable for the coating sleeve to function, as described herein. Further, in some embodiments, the sleeve may include an end terminus that may include an oval or circular lumen that may expand during a guidewire insertion, for example, through GWG 102. The sleeve may also be straight-cut or angled to reduce the total surface area of material around the guidewire, potentially that would enlarge the distal septum during storage post-manufacturing prior to use.

The hypotube may also include a lased sleeve that may be constructed from a flexible alloy, such as nitinol or a grade of stainless steel (with exemplary compositions of 16% to 25% chromium and between 6% to 25% nickel). The lased sleeve may be cut with different patterns and may include a coating for reduced friction, in particular when in contact with distal septum 108. The lased sleeve may also include an end terminus that employs a cylindrical collar to ensure that no snagging on other components occurs. The lased sleeve may include a relatively smaller outer diameter at the end terminus and/or may include segments having different expansion capabilities.

GWG 102 may, in some embodiments, include combinations of expandable sleeves and lased transitions. For example, the hypotube may include a sleeve having both an expandable braided sleeve and a lased transition section. In some embodiments, the hypotube may include a lased expandable sleeve.

The hypotube described herein is more “septum friendly” than at least some known GWG tubes, since the hypotube has a distal tip that is more flexible and/or has a reduced footprint (e.g., smaller outer diameter), reducing friction between the hypotube and other components (e.g. tail, nose, distal septum 108, cannula 110, FAT 112, distal end 114 of the impeller (all shown in FIG. 2), and other components that may be in contact with the hypotube). With the distal tip capable of expanding only temporarily or a minimal amount of time (e.g., for less than thirty seconds during guidewire insertion by the physician), septum sealing efficiency is maintained and a material “memory” of the septum would not be impacted.

Additionally, the hypotube may include diameters or gauge sizes smaller or larger than existing GWG tube outer diameters and/or inner diameters, in order to accommodate guidewires of varied dimensions dependent on coronary, peripheral, neurovascular, biliary, or alternate anatomies. For example, the dimensions of the hypotube may include an outer diameter ranging from 0.0120-0.0125 inches, a wall thickness of approximately 0.002 inches, and an inner diameter ranging from 0.0075-0.0090 inches.

In some embodiments, the hypotube includes a lased sleeve that may be constructed from a flexible alloy, such as nitinol, a grade of stainless steel, or advanced polymers, such as polyamide, HDPE, LLDPE, FEP, PET, Pebax, etc. The lased sleeve may be cut with different patterns and may include a coating for reduced friction, in particular when in contact with distal septum 108. The diameter of the lased sleeve may decrease across distal septum 108, allowing septum 108 to substantially close. In some embodiments, if distal septum 108 includes two or more elastomeric discs, the lased sleeve may also accommodate slight movement and/or deformation of the discs. Once the lased sleeve exits distal septum 108 distally, the lased sleeve expands back out to a larger diameter (which may be the same as or smaller than a diameter of portions of the associated hypotube that are proximal of distal septum 108).

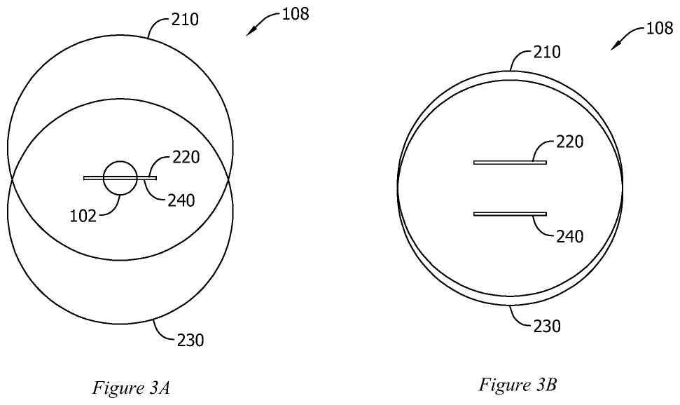

FIG. 3A shows a plan view of septa discs 210 and 230 with aperture alignment. As explained above, with respect to distal septum 108, distal septum 108 includes two separate septa discs. As further explained above, each septa disc 210 and 230 are punctured during catheter assembly by a sharp tool such that a guidewire may be threaded therethrough. In some embodiments, GWG 102 may also be threaded through septa discs 210 and 230.

Septa discs 210 and 230 may consist of a uniform elastomeric material capable of being easily punctured to form an aperture or opening. In some embodiments, more than one elastomeric material may be used. In some cases, two elastomeric materials of varying densities may be used. The specific type of material used may be determined by cost, manufacturing capability, pliability, and flexibility, among other considerations. In some embodiments, septa discs 210 and 230 are made of different materials. In some embodiments, septa discs 210 and 230 may be formed via shells with varying density internally such as a gel or gel-like substance.

Puncture tools of varying widths corresponding to the width of the guidewire and/or GWG 102 may be used to create a sufficient passageway. During storage of distal septum 108, GWG 102 remains in place across distal septum 108. One problem with a long storage shelf life is that for certain materials, such as a silicone elastomer, the material may creep such that when the GWG and/or guidewire are removed, distal septum 108 may not assume its original shape.

FIG. 3B shows a plan view of septa discs with disc alignment. When septa disc 210 and septa disc 230 are placed under lateral compression for positioning inside pump assembly 100 (shown in FIGS. 1 and 2), apertures 220 and 240 are disaligned thereby inhibiting the flow of fluids such as blood into the lumen of pump assembly 100.

To minimize the effect of deformation in septa discs 210 and 230, puncture points at apertures 220 and 240 in each septa disc 210 and 230 through which the guidewire and/or GWG 102 are threaded facilitates improved impedance of fluid flow when septa discs 210 and 230 are positioned within the catheter assembly. As shown in FIGS. 2A and 2B, puncture points may be formed by a tool with a sharp elongated edge and thus appear as a slot or slit. The width of the aperture may be dependent on the size of the guidewire or GWG 102. In the exemplary embodiment, the width of the aperture is sufficient to accommodate deformation of septa discs 210 and 230. In some embodiments, apertures in septa discs 210 and 230 may be formed by tools of varying shapes and sizes determined by the size of the intended guidewire or GWG 102 threaded through the apertures. In some embodiments, apertures 220 and 240 may also include some spatial height. In other embodiments, each side of the aperture remains in contact keeping the aperture in a “closed” position.

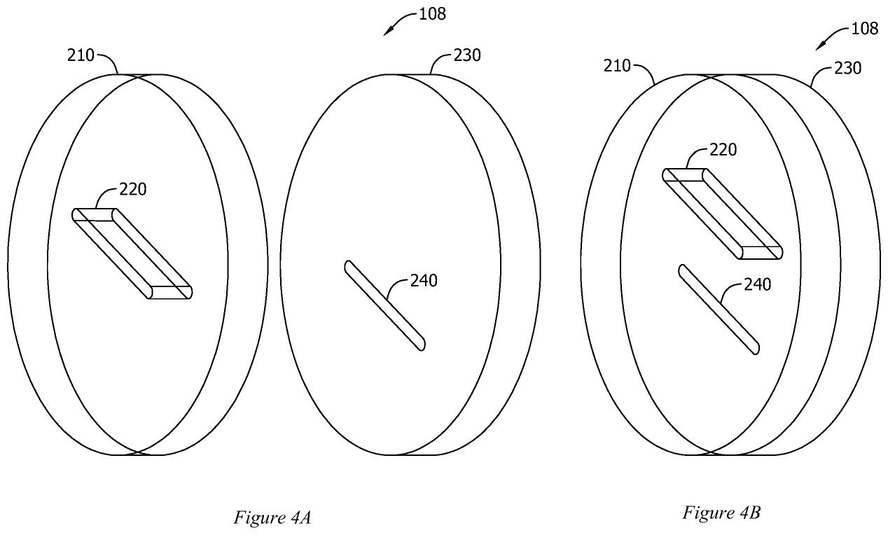

FIG. 4A is a perspective view of septa discs with spatial separation. As shown in FIG. 4A, distal septum 108 includes two septa discs, septa disc 210 and septa disc 230. Septa disc 210 includes an aperture 220 positioned substantially away from aperture 240 on septa disc 230 to minimize the possibility of the flow of fluids through apertures 220 and 240.

FIG. 4B is a perspective view of septa discs conjoined. Upon assembly of pump assembly 100, septa disc 210 is positioned adjacent and substantially in contact with septa disc 230. Accordingly, aperture 220 on septa disc 210 is positioned against the surface of septa disc 230. Any deformities in aperture 220 are thus encompassed by at least a portion of a solid surface or wall of septa disc 230. Similarly, aperture 240 on septa disc 230 is likewise positioned against the surface of septa disc 210. Accordingly, the arrangement inhibits the flow of fluids between septa disc 210 and septa disc 230.

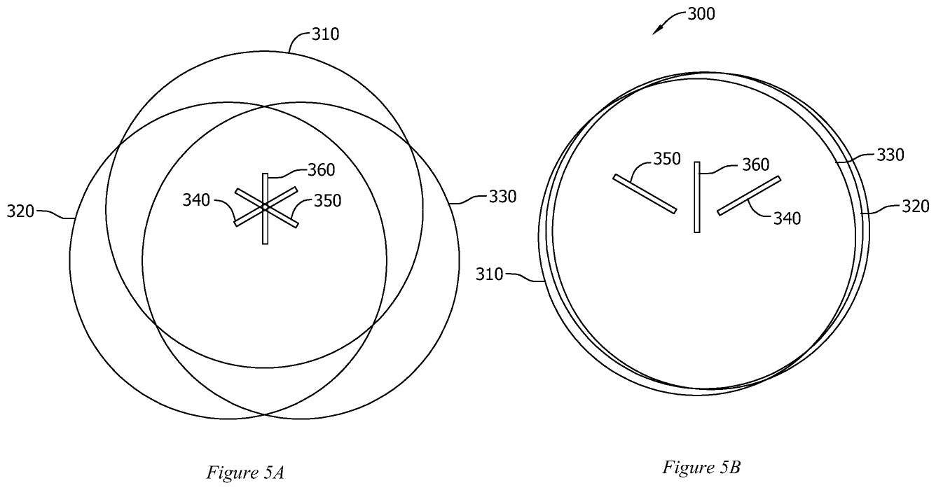

FIG. 5A is a plan view 300 of three septa discs with GWG aperture alignment. To further impede the flow of fluids into the pump assembly 100 (shown in FIGS. 1 and 2), three septa disc may be used to prevent the flow of fluids in pump assembly 100. For example, a first septa disc 310 having an aperture 360 formed by a tool puncturing first septa disc 310, a second septa disc 320 having an aperture 340 formed by a tool puncturing second septa disc 320, and a third septa disc 330 having an aperture 350 are used. Prior to assembly into pump assembly 100, apertures 340, 350, and 360 are in alignment and permit a guidewire or GWG 102 to be threaded through septa discs 310, 320, and 220.

FIG. 5B is a plan view of three septa discs with disc alignment. When placed under lateral compression and assembled into pump assembly 100, apertures 340, 350, and 360 are misaligned or otherwise offset thereby inhibiting the flow of fluids between septa discs 310, 320, and 330. Various arrangements of apertures may be used to prevent fluid flow. Specific arrangements may be based on fluid flow reduction, the potential for deformation of the septa discs, manufacturing costs, tools used to form apertures in the septa discs, the type of material used for the septa discs, among other considerations.

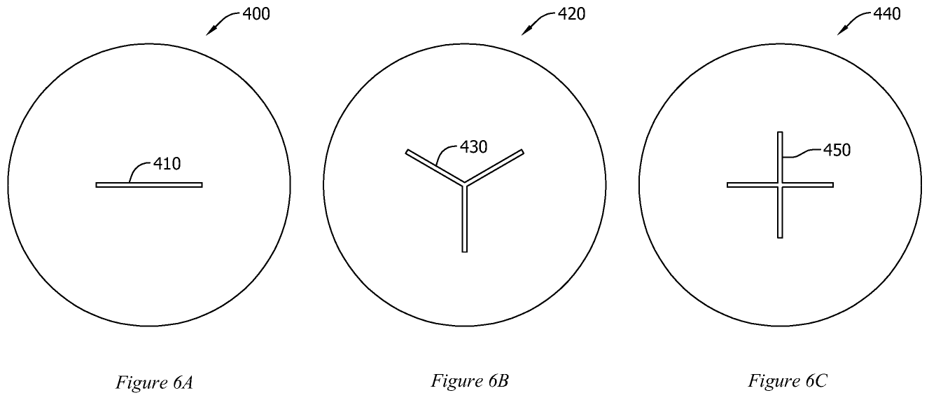

FIG. 6A illustrates one embodiment of a GWG aperture in a line pattern. As explained above, a puncture tool may be used to form passageways through which a guidewire or guidewire guide may be placed. In the exemplary embodiment, the puncture tool may be an elongated, sharp-edged blade designed to puncture septa disc 400 in a horizontal fashion. In some embodiments, multiple punctures may be performed and/or a tool having varying formations may be used.

FIG. 6B illustrates an aperture in a Y pattern. Septa disc 420 may be punctured through the use of a single tool having an angled Y-shaped or similar pattern. Alternatively, multiple cuts may be made in septa disc 420 to form aperture 430. Each cut may include a distal end converging upon a single point in septa disc 420.

FIG. 6C illustrates an aperture in a cross pattern. In embodiments where multiple cuts are performed, aperture 450 on septa disc 440 may be formed by overlapping cuts. Alternative patterns may be used to achieve the prevention of fluid flow.

The embodiments described herein provide systems and methods for guidewire guides in implantable medical devices. Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A septum for use in a catheter pump, the septum comprising a first elastomeric septa disc punctured to form an aperture in the first elastomeric septa disc, the aperture passable by a guidewire.

2. The septum of claim 1, wherein the septum includes a second elastomeric septa disc having an aperture, the aperture of the second elastomeric septa disc positioned in alignment with the aperture of the first elastomeric septa disc prior to placement of the first elastomeric septa disc and second elastomeric septa disc in the catheter pump.

3. The septum of claim 2, wherein when the first elastomeric septa disc and second elastomeric septa disc are positioned within the catheter pump, the aperture of the first elastomeric septa disc and the aperture of the second elastomeric septa disc are offset.

4. The septum of claim 2, wherein the septum includes a third elastomeric septa disc having an aperture, the aperture of the third elastomeric septa disc positioned in alignment with the aperture of the first elastomeric septa disc and in alignment with the second elastomeric septa disc prior to the placement of the first elastomeric septa disc, second elastomeric septa disc, and third elastomeric septa disc in the catheter pump.

5. The septum of claim 4, wherein when the first elastomeric septa disc, second elastomeric septa disc, and third elastomeric septa disc are positioned within the catheter pump, the aperture of the first elastomeric septa disc, the aperture of the second elastomeric septa disc, and the aperture of the third elastomeric septa disc are offset with each other respective aperture.

6. The septum of claim 1, wherein the aperture of the first elastomeric septa disc is formed by a tool capable of forming an aperture in the first elastomeric septa disc by a single cut.

7. The septum of claim 6, wherein the aperture of the first elastomeric septa disc is formed by at least a first cut by the tool and a second cut by the tool, the first cut overlapping the second cut.

8. A catheter assembly for use in a percutaneous heart pump, the catheter assembly comprising:
a pump assembly comprising:
an impeller;
an impeller tip positioned distal of the impeller; and
a distal septum positioned between the impeller and the impeller tip;
a flexible atraumatic tip (FAT) positioned distal of the pump assembly; and
a guidewire guide (GWG) coupled between the pump assembly and the FAT, wherein a proximal end of the GWG is positioned distal of the distal septum.

9. The catheter assembly of claim 8, wherein the distal septum comprises a first elastomeric septa disc, the first elastomeric septa disc having an aperture passable by a guidewire.

10. The catheter assembly of claim 9, wherein the aperture of the first elastomeric septa disc is aperture passable by the GWG.

11. The catheter assembly of claim 9, wherein the distal septum includes a second elastomeric septa disc having an aperture, the aperture of the second elastomeric septa disc positioned in alignment with the aperture of the first elastomeric septa disc prior to placement of the first elastomeric septa disc and second elastomeric septa disc in the pump assembly.

12. The catheter assembly of claim 9, wherein when the first elastomeric septa disc and second elastomeric septa disc are positioned within the catheter assembly, the aperture of the first elastomeric septa disc and the aperture of the second elastomeric septa disc are offset.

13. The catheter assembly of claim 9, wherein the aperture of the first elastomeric septa disc is formed by a tool capable of puncturing the first elastomeric septa disc, the puncture forming an aperture in the first elastomeric septa disc by a first cut.

14. The catheter assembly of claim 13, wherein the aperture of the first elastomeric septa disc is formed by a second cut overlapping the first cut.

15. A method of assembling a catheter assembly for use in a percutaneous heart pump, the method comprising:
coupling a pump assembly to the catheter assembly, wherein assembling the pump assembly comprises:
positioning an impeller in the pump assembly;
positioning an impeller tip in the pump assembly at a position distal of the impeller; and
positioning a distal septum between the impeller and the impeller tip;
positioning a flexible atraumatic tip (FAT) distal of the pump assembly; and
coupling a guidewire guide (GWG) between the pump assembly and the FAT, wherein a proximal end of the GWG is positioned distal of the distal septum.

16. The method of claim 15 further comprising assembling the distal septum, wherein assembling the distal septum comprises:
puncturing a first elastomeric septa disc to form an aperture in the first elastomeric septa disc;
puncturing a second elastomeric septa disc to form an aperture in the second elastomeric septa disc;
aligning the aperture of the first elastomeric septa disc with the aperture of the second elastomeric septa disc;
threading a guidewire through the aperture of the first elastomeric septa disc;
threading the guidewire through the second elastomeric septa disc;
applying lateral compression on the first elastomeric septa disc and the second elastomeric septa disc, wherein the lateral compression substantially aligns the first elastomeric septa disc and the second elastomeric septa disc, and wherein the aperture of the first elastomeric septa disc and the aperture of the second elastomeric septa disc are offset; and
positioning the first elastomeric septa disc and the second elastomeric septa disc in the pump assembly between the impeller and the impeller tip.

17. The method of claim 16, wherein positioning the first elastomeric septa disc and the second elastomeric septa disc in the pump assembly comprises positioning the first elastomeric septa disc against the second elastomeric septa disc wherein a first wall of the first elastomeric septa disc is substantially in contact with a first wall of the second elastomeric septa disc.

18. The method of claim 16 further comprising positioning the GWG through the aperture of the first elastomeric septa disc and the second elastomeric septa disc.

19. The method of claim 16, wherein first elastomeric septa disc is punctured by a tool capable of puncturing the first elastomeric septa disc, the tool having a shape and size capable of forming the aperture in the first elastomeric septa disc, the aperture having a size and shape sufficient to permit passage of a guidewire.

20. The method of claim 16, wherein the aperture of the first elastomeric septa disc is formed by a first cut in the first elastomeric septa disc and a second cut in the first elastomeric septa disc, wherein the second cut in the first elastomeric septa disc overlaps the first cut in the first elastomeric septa disc.
